(19) United States
(12) Patent Application Publication
(10) Pub. No.: US 2014/0051141 A1
(43) Pub. Date: Feb. 20, 2014

(54) METHOD FOR INCREASING ALCOHOL YIELD FROM GRAIN

(71) Applicant: Arisdyne Systems, Inc., Cleveland, OH (US)

(72) Inventors: Oleg KOZYUK, North Ridgeville, OH (US); Peter REIMERS, Shaker Heights, OH (US)

(73) Assignee: Arisdyne Systems, Inc., Cleveland, OH (US)

(21) Appl. No.: 13/964,373

(22) Filed: Aug. 12, 2013

Related U.S. Application Data
(60) Provisional application No. 61/682,886, filed on Aug. 14, 2012.

(51) Int. Cl. C12P 7/14

(52) U.S. Cl. CPC C12P 7/14
USPC 435/162

(57) ABSTRACT

A process for increasing alcohol yield from grain that includes adding cellulase enzymes or a mixture of cellulase enzymes to break down cellulostic feedstock, which is typically discarded. The cellulase enzymes or a mixture of cellulase enzymes may be added to a conventional alcohol production process either through a joint or separate fermentation process.

This application claims the benefit of U.S. provisional application Ser. No. 61/682,886 filed Aug. 14, 2012, the contents of which are incorporated herein in their entirety by reference.

### FIELD

The invention relates to processes for producing alcohol, and more particularly, processes for increasing alcohol yield using by-products typically discarded from conventional alcohol operations.

### BACKGROUND

Alcohols are a renewable and clean fuel source. A grain alcohol commonly used as a fuel source is ethanol, which can be produced, in large part, from corn by the fermentation of starch. Generally, alcohol production is accomplished through a fermentation and distillation process wherein starches are released and converted to sugars, and then the sugars are converted to alcohol by the addition of yeast. At an industrial level, yeast fermentation processes only convert about one-third of the corn into alcohol.

Alcohol production facilities often begin the production process with a dry or wet milling process. In dry milling, corn, or another suitable grain, is ground up by a hammer or roller mill into a dry mixture of particles. The dry mixture of particles is combined with water and enzymes to break up the starch from the corn into smaller fragments and then subject the smaller fragments to a saccharification phase wherein the starch is converted to sugar. After the saccharification phase, resulting sugars are fermented with yeast to facilitate their conversion to alcohol.

Alcohol yield is dependent upon initial starch content of corn as well as the availability of starch to enzymes that are used in the saccharification phase. In conventional processes, the availability of starch is governed, in part, by the success of the dry milling or similar step in which the corn is broken up into smaller particles. Production processes currently used in commercial alcohol plants are not able to achieve maximum theoretical alcohol yield, which results in a significant amount of lost and discarded starch in the form of by-products such as Distiller's Dried Grains with Solubles (DDGS). Accordingly, there is still a need for a process that can obtain a closer to theoretical maximum yield to produce a certain amount of alcohol.

### SUMMARY

A process for producing alcohol that includes the steps of mixing grain and water to create a slurry that includes starch and cellulostic feedstock containing cellulose and hemi-cellulose. The starch in the slurry is hydrolyzed with non-cellulase enzymes to create a mixture including a starch hydrolysate and a non-hydrolyzed cellulostic feedstock that is insoluble in the starch hydrolysate. The non-hydrolyzed cellulostic feedstock is partially hydrolyzed in a holding tank for at least 0.1 hour with a cellulase enzyme or a mixture of cellulase enzymes to create a partially hydrolyzed cellulostic feedstock. The starch hydrolysate and the partially hydrolyzed cellulostic feedstock are fermented with yeast to produce alcohol.

A process for producing alcohol that includes the steps of mixing grain and water to create a slurry that includes starch and cellulostic feedstock containing cellulose and hemi-cellulose. The starch in the slurry is hydrolyzed with non-cellulase enzymes to create a mixture comprising a starch hydrolysate and a non-hydrolyzed cellulostic feedstock that is insoluble in the starch hydrolysate. A portion of the non-hydrolyzed cellulostic feedstock is separated from the starch hydrolysate and the separated non-hydrolyzed cellulostic feedstock is transferred into a holding tank. The starch hydrolysate is fermented with yeast to produce alcohol. The non-hydrolyzed cellulostic feedstock is partially hydrolyzed in the holding tank for at least 0.1 hour with a cellulase enzyme or a mixture of cellulase enzymes to create a partially hydrolyzed cellulostic feedstock that is fermented with yeast to produce alcohol.

### BRIEF DESCRIPTION OF THE DRAWINGS

FIG. 1 is a flow diagram of a conventional alcohol production process.

FIG. 2 is a flow diagram of an alcohol production process using cellulase enzymes or a mixture of cellulase enzymes in a single fermentation process. The cellulase enzymes or a mixture of cellulase enzymes are added to a holding tank to partially hydrolyze cellulostic feedstock.

FIG. 3 is a flow diagram of an alcohol production process using cellulase enzymes or a mixture of cellulase enzymes in a single fermentation process. The cellulase enzymes or a mixture of cellulase enzymes are added to a holding tank to partially hydrolyze cellulostic feedstock that has been subjected to secondary milling.

FIG. 4 is a flow diagram of an alcohol production process using cellulase enzymes or a mixture of cellulase enzymes in a single fermentation process. The cellulase enzymes or a mixture of cellulase enzymes are added to a holding tank to partially hydrolyze cellulostic feedstock wherein the cellulase enzymes or a mixture of cellulase enzymes and cellulostic feedstock are subjected to secondary milling.

FIG. 5 is a flow diagram of an alcohol production process using cellulase enzymes or a mixture of cellulase enzymes in a single fermentation process. The cellulase enzymes or a mixture of cellulase enzymes are added to a holding tank to partially hydrolyze cellulostic feedstock that has been separated from starch hydrolysate and subjected to secondary milling.

FIG. 6 is a flow diagram of an alcohol production process using cellulase enzymes or a mixture of cellulase enzymes in a separate fermentation process. The cellulase enzymes or a mixture of cellulase enzymes are added to a holding tank to partially hydrolyze cellulostic feedstock that has been separated from starch hydrolysate and subjected to secondary milling.

### DETAILED DESCRIPTION

Herein, when a range such as 5-25 (or 5 to 25) is given, this means preferably at least or more than 5 and, separately and independently, preferably not more than or less than 25. In an example, such a range defines independently not less than 5, and separately and independently, not more than 25.

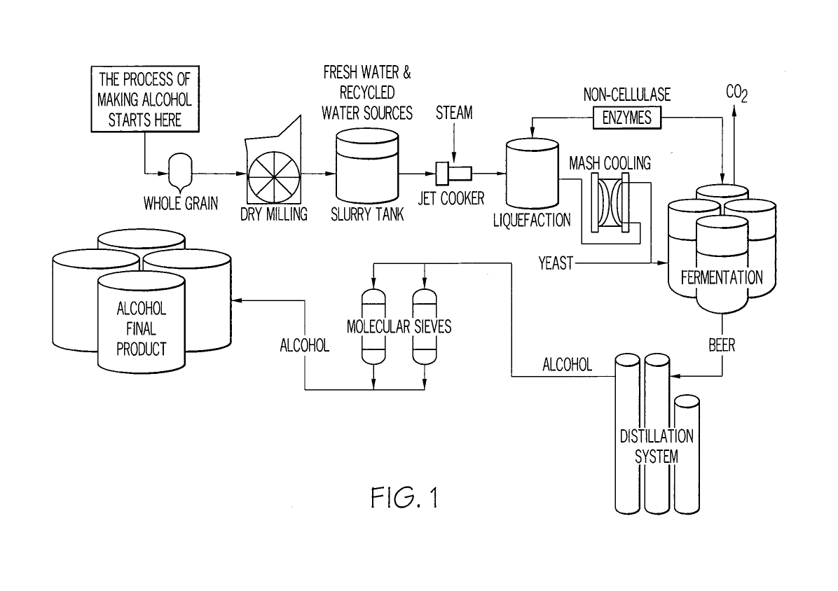

FIG. 1 shows a conventional alcohol production process, such as that used to produce ethanol, for example, a starch-to-ethanol production process. The alcohol production process shown utilizes a first milling means, such as a dry milling step, to grind grain, such as whole kernel corn, into meal or powder. Preferably, the grain is screened to remove foreign material or debris, such as dirt, stalks, leaves and the like. Although corn is shown as the whole grain in FIG. 1, any grain can be used. For example, grains can include corn, rye, sorghum, wheat, beans, barley, oats, rice, or combinations thereof. As used herein, the term “grain” can comprise whole grain or portions or particles of whole grains such as the product from a dry- or wet-milling process used in an alcohol production process. The ground grain powder is combined with a fluid carrier, such as water, to make a slurry comprising starch and cellulostic feedstock, which contains cellulose, hemi-cellulose, lignin and protein. Additional ground corn kernels and cellulose fibers can be optionally added to the slurry. The slurry comprises preferably at least 5, 10, 15, 20, 25, 30, 40, 50 or 60 weight percent grain, based on the total weight of the slurry in a slurry tank. As shown in FIG. 1, the slurry comprises grains and a liquid carrier, such as water.

The slurry can be heated in a cooking phase, such as by a jet cooker, at approximately 93 to 95 degrees Celsius or above and at 10 to 40 psi. The slurry can be subsequently held at an elevated temperature of about 80 to 90 degrees Celsius for a period of about 4 to 8 hours. Alternatively, the temperatures, pressures and time periods noted above can vary widely depending on a specific application. The jet cooker and the subsequent heating period preferably solubilize the starch contained the in grains in the fluid carrier.

In the alcohol production process, a liquefaction phase follows the cooking phase, at which point non-cellulase enzymes are added to the slurry in order to break down the starch polymer into short sections. Non-cellulase enzymes may include α-Amylase, β-Amylase, and γ-Amylase enzymes. The non-cellulase enzymes are preferably added between 50 to 60 degrees Celsius. The non-cellulase enzymes typically do not effectively hydrolyze starch at conditions, such as temperature, that cellulase enzymes are most effective. The liquefaction phase produces a hydrolyzed mixture from the slurry comprising a starch hydrolysate and a non-hydrolyzed cellulostic feedstock, which is insoluble in the starch hydrolysate. The starch hydrolysate includes conventional starch to be fermented, while the non-hydrolyzed cellulostic feedstock can include cellulose, hemicellulose, lignin and protein that would typically be discarded as waste materials. The non-hydrolyzed cellulostic feedstock is at least 50 weight percent solid, and the short sections can be maltodextrins and oligosaccharides. A saccharification phase follows the liquefaction phase. The non-cellulase enzymes in the saccharification phase create a sugar mash in a mash cooling phase that can be transferred into fermentation tanks where yeast can convert sugars into carbon dioxide and alcohol, such as ethanol. In addition to alcohol, soluble and insoluble solids, which can include non-fermentable components and cellulostic feedstock, are left over from the grain. A distillation phase following the fermentation phase separates the liquid carrier, usually water, ethanol, and whole stillage. The water can be recycled and used, for example, in the slurry tanks. The cellulostic feedstock is further separated in the distillation process, and can also be sold as high-protein animal feed.

As described, a significant amount of cellulostic feedstock is lost and discarded in the form of by-products such as Distiller's Dried Grains with Solubles (DDGS). DDGS typically contains about 12-15% cellulose and hemicellulose by weight on a dry weight basis, to which about 4-10% by weight starch can be bound. These by-products are not typically broken down or hydrolyzed by non-cellulase enzymes in conventional alcohol production. However, utilizing cellulase enzymes or a mixture of cellulase enzymes reduces the amount discarded by recovering glucose, xylose and arabinose from cellulose and hemicellulose. The cellulase enzymes or the mixture of cellulase enzymes comprises cellulases, xylanases or ligninases. As a result, as described below, the addition of the cellulase enzymes or mixture of cellulase enzymes can partly hydrolyze cellulostic feedstock prior to any fermentation steps and convert cellulose in the feedstock into glucose and hemicellulose in the feedstock into xylose and arabinose that can be subsequently fermented with yeast to produce alcohol. Preferably, less than 50, 40, 30, 20, 10, 5, 4, 3, 2 or 1 weight percent of the cellulostic feedstock is hydrolyzed by the cellulase enzymes or mixture of cellulase enzymes. The use of cellulase enzymes or a mixture of cellulase enzymes can increase and improve alcohol yield over conventional alcohol processing. As described below, the cellulase enzymes or a mixture of cellulase enzymes can be added at a concentration of 0.015 to 0.5 weight percent by weight of grain, such as corn. For example, the cellulase enzymes or mixture of cellulase enzymes can be added at a concentration of at least 0.015, 0.016, 0.2, 0.28, 0.3, 0.4 or 0.5 weight percent.

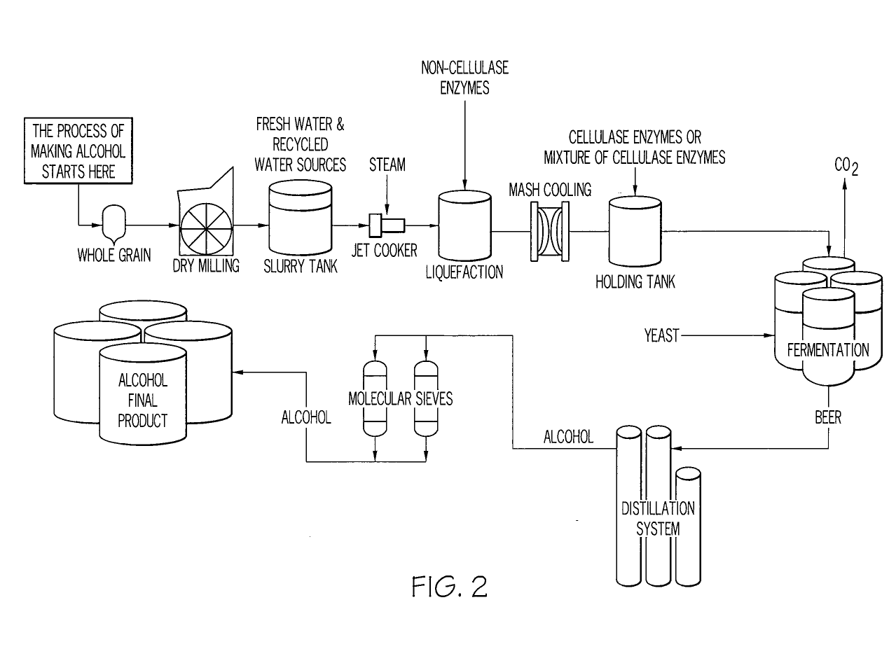

As shown in FIG. 2, steps from the conventional alcohol production process as shown in FIG. 1 may be followed to the liquefaction phase where the non-cellulase enzymes are added to the mixture to breakdown and hydrolyze the starch in the mixture to create a starch hydrolysate. After the liquefaction phase, the mixture containing the starch hydrolysate and a non-hydrolyzed cellulostic feedstock that is insoluble in the starch hydrolysate may flow through the mash cooler that cools the mixture from 80 to 95 degrees Celsius to below 55 degrees Celsius. Following the mash cooling phase, the mixture may enter a holding tank where cellulase enzymes or the mixture of cellulase enzymes may be added to break down the non-hydrolyzed cellulostic feedstock. The mixture may be in the holding tank for a period of 0.1 to 4 or 2 to 24 hours at a pH of 4.0-5.5. The mixture can be maintained in the holding tank at a temperature of 30 to 55 degrees Celsius where the cellulase enzymes or the mixture of cellulase enzymes are suitable for carrying out a partial hydrolysis reaction as compared to non-cellulase enzymes that are more effective at elevated temperatures above 50 degrees Celsius, such as that experienced in the cooking phase. While in the holding tank, the cellulase enzymes or the mixture of cellulase enzymes partially hydrolyze the non-hydrolyzed cellulostic feedstock by a hydrolysis reaction to produce a partially hydrolyzed cellulostic feedstock. Following the holding tank, the starch hydrolysate and partially hydrolyzed cellulostic feedstock may be fermented together with yeast to produce alcohol.

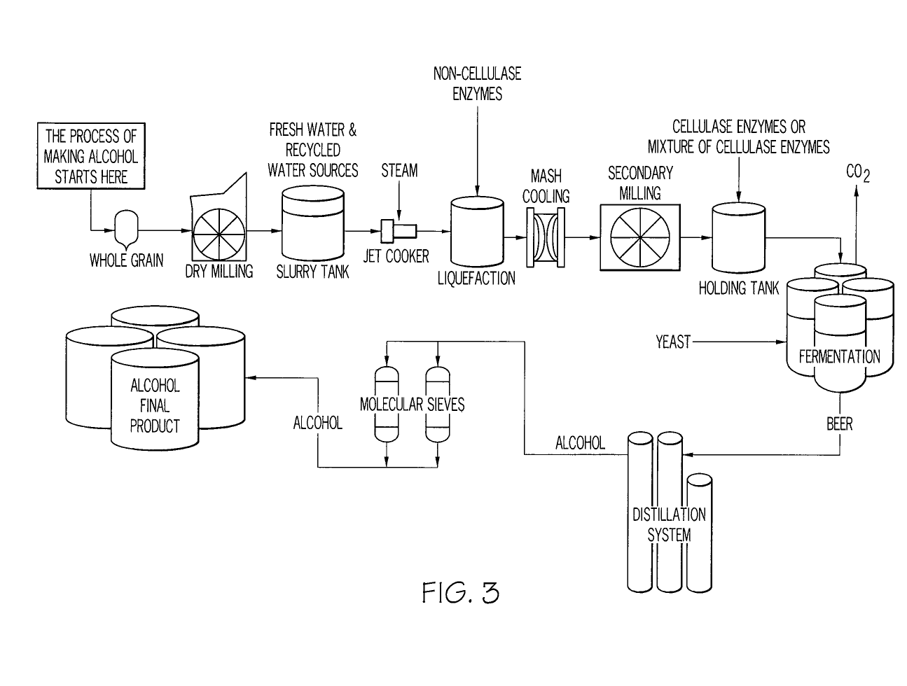

In addition to the flow diagram of FIG. 2, the mixture exiting the mash cooling phase may undergo a secondary milling phase as shown in FIG. 3 prior to being transferred to the holding tank. Following the addition of the non-cellulase enzymes to the mixture in the liquefaction phase, the mixture may pass through the mash cooler. Following the mash cooler, the mixture may be treated with a secondary milling means to further break down the non-hydrolyzed cellulostic feedstock in the mixture. The secondary milling means provides access to the non-hydrolyzed cellulostic feedstock by producing a colloidal suspension of biomass, which allows the cellulase enzymes or mixture of cellulase enzymes better access to hydrolyze the cellulose and hemi-cellulose in the biomass that was not hydrolyzed in the mixture during the liquefaction phase. The secondary milling means may be rotatory mixers, rotary milling devices, rotor-rotor and rotor-stator devices, media and attrition milling devices, disc and impact mills, jet mixers, homogenizer, hydrodynamic or ultrasonic cavitation devices or combination thereof.

Following the secondary milling means, cellulase enzymes or the mixture of cellulase enzymes may be added to the mixture to break down and partially hydrolyze the non-hydrolyzed cellulostic feedstock in the holding tank, wherein the non-hydrolyzed cellulostic feedstock is held and mixed for at least 0.1 hour. The cellulase enzymes or the mixture of cellulase enzymes are preferably added at a temperature of 30 to 55 degrees Celsius and at the pH of 4.0 to 5.5 for a period of 0.1 to 4 or 2 to 24 hours. Following the holding tank, the starch hydrolysate and partially hydrolyzed cellulostic feedstock may be fermented jointly with yeast to produce alcohol.

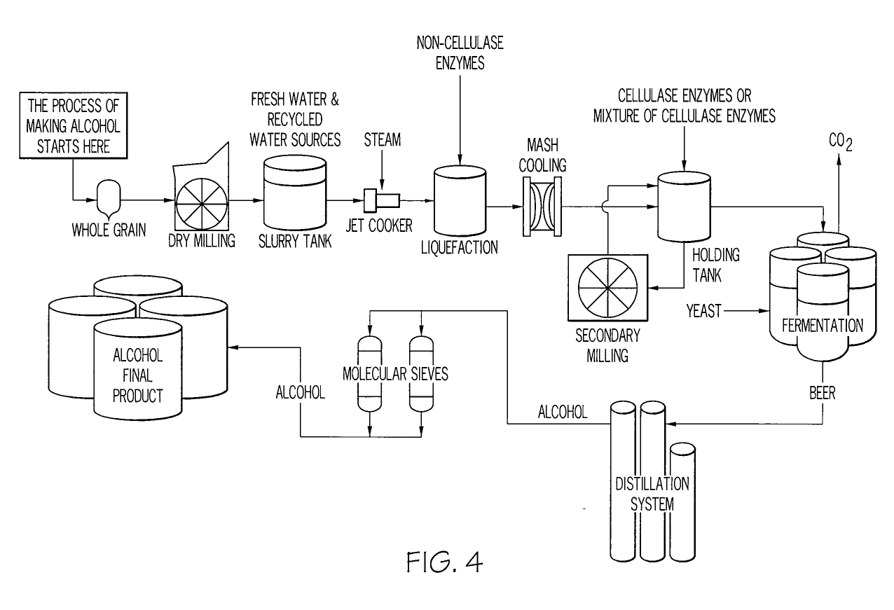

Alternatively to FIG. 3, the mixture of starch hydrolysate and non-hydrolyzed cellulostic feedstock may enter the holding tank following the mash cooler and prior to the secondary milling means as shown in FIG. 4. Cellulase enzymes or the mixture of cellulase enzymes may be added to the holding tank to partially hydrolyze a portion of the non-hydrolyzed cellulostic feedstock to produce a partially hydrolyzed cellulostic feedstock prior to any fermentation steps. Once in the holding tank, the starch hydrolysate, the partially hydrolyzed cellulostic feedstock and remaining portions of the non-hydrolyzed cellulostic feedstock and cellulase enzymes or the mixture of cellulase enzymes may enter the secondary milling means and re-enter the holding tank by use of a recirculation loop. The cellulase enzymes or mixture of cellulase enzymes may be added into the holding tank for 0.1 to 4 or 2 to 24 hours at 30 to 55 degrees Celsius and at the pH of 4.0-5.5. Following the holding tank, the starch hydrolysate and the partially hydrolyzed cellulostic feedstock may be fermented jointly with yeast to produce alcohol.

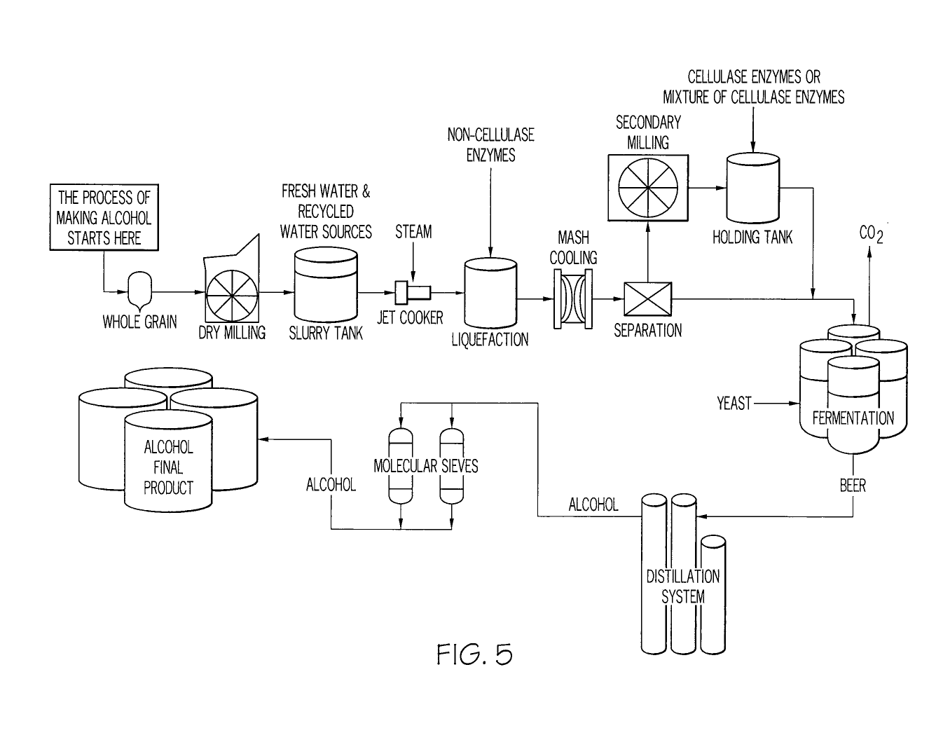

In another embodiment, the mixture exiting the mash cooling phase, preferably below 55, 50, 45 or 40 degrees Celsius, may be separated into starch hydrolysate stream and a non-hydrolyzed cellulostic feedstock stream as shown in FIG. 5. Following the liquefaction phase, where the non-cellulase enzymes are added, the mixture may enter the mash cooling phase. Following mash cooling, a separation method may be used to separate the starch hydrolysate from the non-hydrolyzed cellulostic feedstock in the mixture such that the starch hydrolysate can be transferred to a fermentation phase and the non-hydrolyzed cellulostic feedstock can be transferred to a separate holding tank. A separation phase may include centrifuges, cyclones, paddle screens, or gravity and pressure screens. Separation of the two streams may even be carried out by a combination of the separating methods described above.

Once separated, the non-hydrolyzed cellulostic feedstock may enter the secondary milling means to break down the non-hydrolyzed cellulostic feedstock. Following the secondary milling means, the cellulase enzymes or mixture of cellulase enzymes may be added to the non-hydrolyzed cellulostic feedstock in the holding tank to create the partially hydrolyzed cellulostic feedstock. The non-hydrolyzed cellulostic feedstock may be in the holding tank for the period of 0.1 to 4 or 2 to 24 hours at 30 to 55 degrees Celsius and at the pH of 4.0 to 5.5. Following the holding tank, the partially hydrolyzed cellulostic feedstock may be combined with the starch hydrolysate for joint fermentation with yeast to produce alcohol.

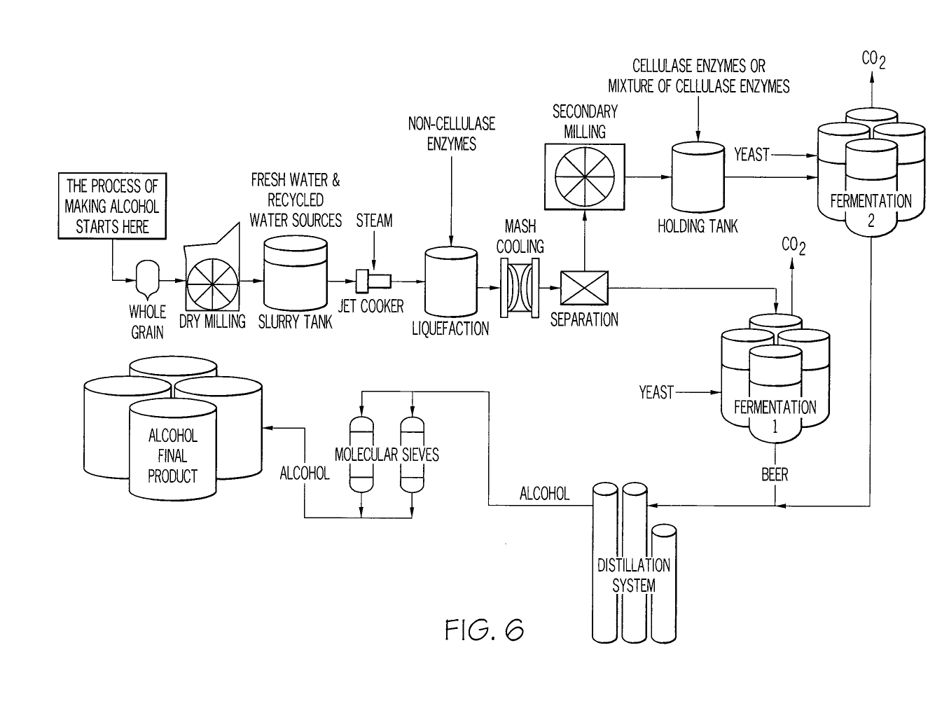

In another embodiment, the starch hydrolysate and partially hydrolyzed cellulostic feedstock may be fermented in separate fermentation operations as shown in FIG. 6. Similar to FIG. 5, the slurry enters the liquefaction phase with non-cellulase enzymes to create a mixture of starch hydrolysate and non-hydrolyzed cellulostic feedstock. The mixture may enter the mash cooling phase, which is followed by separating the starch hydrolysate and non-hydrolyzed cellulostic feedstock. The starch hydrolysate may be fermented under its own fermentation operation with yeast as shown in FIG. 6. Following the separation, the non-hydrolyzed cellulostic feedstock may optionally enter the secondary milling means. The milled non-hydrolyzed cellulostic feedstock can be transferred to a holding tank where the cellulase enzymes or mixture of cellulase enzymes may be added to induce a partial hydrolysis reaction to create the partially hydrolyzed cellulostic feedstock. For single fermentation operations, the cellulase enzymes or mixture of cellulase enzymes may be in the holding tank for 0.1 to 4 hours at 30 to 55 degrees Celsius and at the pH of 4.0-5.5, which may be followed by the partially hydrolyzed cellulostic feedstock entering into its own separate fermentation operation with yeast to produce alcohol.

In addition to the methods described above, a controlled flow cavitation apparatus may be used as the secondary milling means to apply a specified cavitation activation energy. From the methods described above, hydrolyzed or non-hydrolyzed cellulostic feedstock and the cellulase enzymes or mixture of cellulase enzymes may pass through the controlled flow cavitation apparatus. Alternatively, the cellulase enzymes or mixture of cellulase enzymes without partially hydrolyzed or non-hydrolyzed cellulostic feedstock may pass through the controlled flow cavitation apparatus. Similarly, the non-cellulase enzymes may pass through a controlled flow cavitation apparatus with or without a mixture of starch and cellulostic feedstock. In addition, a mixture of starch and cellulostic feedstock, cellulase enzymes, and non-cellulase enzymes may enter together through the controlled flow cavitation apparatus.

Examples of static cavitational energy sources that can be used to apply cavitational energy to the non-hydrolyzed cellulostic feedstock include, but are not limited to, static mixers, orifice plates, perforated plates, nozzles, venturis, jet mixers, eductors, cyclonettes (e.g., Fluid-Quip, Inc.), and control flow cavitation devices (e.g., Arisdyne systems, Inc.), such as those described in U.S. Pat. Nos. 5,810,052; 5,931,771; 5,937,906; 5,971,601; 6,012,492; 6,502,979; 6,802,639; 6,857,774 and 7,667,082. Additionally, the dynamic cavitational energy sources that can be used include, but are not limited to, rotary milling devices (e.g., EdeniQ Cellunator™), rotary mixers (e.g., HydroDynamics SPR, Magellan™), rotor-rotor (e.g., Eco-Fusion Canada Inc.) and rotor-stator devices (e.g., IKA® Works, Inc., Charles Ross & Son Company, Silverson Machines, Inc., Kinematica Inc.), such as those described in U.S. Pat. Nos. 6,857,774; 7,178,975; 5,183,513; 5,184,576; 5,239,948; 5,385,298; 5,957,122; and 5,188,090.

It should now be apparent that there has been provided, in accordance with the present invention, a novel process for enhancing alcohol production by utilizing conventional starch by-products that satisfies the benefits and advantages set forth above. Moreover, it will be apparent to those skilled in the art that many modifications, variations, substitutions and equivalents for the features described above may be effected without departing from the spirit and scope of the invention. Accordingly, it is expressly intended that all such modifications, variations, substitutions and equivalents which fall within the spirit and scope of the invention as defined in the appended claims to be embraced thereby.

The preferred embodiments have been described, herein. It will be apparent to those skilled in the art that the above methods may incorporate changes and modifications without departing from the general scope of this invention. It is intended to include all such modifications and alterations in so far as they come within the scope of the appended claims or the equivalents thereof.

What is claimed is:

1. A process for producing alcohol, comprising:
(a) mixing grain and water to create a slurry comprising starch and cellulostic feedstock containing cellulose and hemi-cellulose;
(b) hydrolyzing the starch in the slurry with non-cellulase enzymes to create a mixture comprising a starch hydrolysate and a non-hydrolyzed cellulostic feedstock that is insoluble in the starch hydrolysate;
(c) partially hydrolyzing the non-hydrolyzed cellulostic feedstock in a holding tank for at least 0.1 hour with a cellulase enzyme or a mixture of cellulase enzymes to create a partially hydrolyzed cellulostic feedstock; and
(d) fermenting the starch hydrolysate and the partially hydrolyzed cellulostic feedstock with yeast to produce alcohol.

2. The process of claim 1, wherein step (c) is carried out before the fermentation phase in step (d) and the mixture of step (b) is subjected to a mash cooling step before step (c), wherein the mixture of step (b) is cooled below 55 degrees Celsius by the mash cooling.

3. The process of claim 1, wherein the grain of step (a) is treated with a first milling means and during or before step (c) the non-hydrolyzed cellulostic feedstock has been additionally treated with a second milling means.

4. The process of claim 3, wherein the second milling means is selected from a group consisting of rotary mixers, rotary milling devices, rotor-rotor and rotor-stator devices, media and attrition milling devices, disc and impact mills, jet mixers, homogenizer, hydrodynamic and ultrasonic cavitation devices.

5. The process of claim 1, wherein the cellulase enzyme or mixture of cellulase enzymes of step (c) partially convert cellulose into glucose and hemicellulose to xylose and arabinose.

6. The process of claim 1, wherein partially hydrolyzing the non-hydrolyzed cellulostic feedstock in step (c) is carried out at a temperature between 30 to 55 degrees Celsius for 0.1 to 4 hours.

7. The process of claim 1, wherein said cellulase enzyme or mixture of cellulase enzymes comprises cellulases, xylanases or ligninases.

8. A process for producing alcohol, comprising:
(a) mixing grain and water to create a slurry comprising starch and cellulostic feedstock containing cellulose and hemi-cellulose;
(b) hydrolyzing the starch in the slurry with non-cellulase enzymes to create a mixture comprising a starch hydrolysate and a non-hydrolyzed cellulostic feedstock that is insoluble in the starch hydrolysate;
(c) separating a portion of the non-hydrolyzed cellulostic feedstock from the starch hydrolysate and transferring the non-hydrolyzed cellulostic feedstock into a holding tank;
(d) fermenting the starch hydrolysate by yeast to produce alcohol;
(e) partially hydrolyzing the non-hydrolyzed cellulostic feedstock in the holding tank for at least 0.1 hour with a cellulase enzyme or a mixture of cellulase enzymes in the holding tank to create a partially hydrolyzed cellulostic feedstock; and
(f) fermenting the partially hydrolyzed cellulostic feedstock by yeast to produce alcohol.

9. The process of claim 8, wherein the mixture of step (b) is subjected to a mash cooling step before step (c), wherein the mixture of step (b) is cooled below 55 degrees Celsius by the mash cooling.

10. The process of claim 8, wherein ground corn kernels or cellulose fibers are added to said grain.

11. The process of claim 8, wherein separation step (c) includes the use of a centrifuge, cyclone, paddle screen, gravity, pressure screen or a combination thereof.

12. The process of claim 8, wherein the cellulase enzyme or mixture of cellulase enzymes and non-hydrolyzed cellulostic feedstock are passed through a controlled flow cavitation apparatus prior to fermentation.

13. The process of claim 8, wherein steps (d) and (f) are carried out as two separate fermentation operations.

14. The process of claim 8, wherein steps (d) and (f) are carried out as one joint fermentation operation.

15. The process of claim 8, wherein the grain of step (a) is treated with a first milling means and during or before step (e) the separated non-hydrolyzed cellulostic feedstock has been treated with a second milling means.

16. The process of claim 15, wherein the second milling means is selected from the group consisting of rotary mixers, rotary milling devices, rotor-rotor and rotor-stator devices, media and attrition milling devices, disc and impact mills, jet mixers, homogenizer, hydrodynamic and ultrasonic cavitation devices.

17. The process of claim 8, wherein the cellulase enzyme or mixture of cellulase enzymes of step (e) convert cellulose into glucose and hemicellulose to xylose and arabinose.

18. The process of claim 8, wherein said cellulase enzyme or mixture of cellulase enzymes comprises cellulases, xylanases or ligninases.

19. The process of claim 8, wherein the separated non-hydrolyzed cellulostic feedstock of step (c) has at least 50 weight percent solids.

20. The process of claim 8, wherein partially hydrolyzing the non-hydrolyzed cellulostic feedstock in step (e) is carried out at a temperature between 30 to 55 degrees Celsius for 0.1 to 4 hours.
